(19) United States
(12) Patent Application Publication
(10) Pub. No.: US 2023/0301556 A1
(43) Pub. Date: Sep. 28, 2023

(54) CONTINUOUS GLUCOSE SENSOR AND MOUNTING ASSEMBLY

(71) Applicant: Tingo Medical Ltd., Tel Aviv (IL)

(72) Inventors: Lior STRAM, Tel Aviv (IL); Ofer YODFAT, Modi'in (IL)

(73) Assignee: Tingo Medical Ltd., Tel Aviv (IL)

(21) Appl. No.: 18/023,322

(22) PCT Filed: Aug. 26, 2021
(86) PCT No.: PCT/IL2021/051052

§ 371 Date: Feb. 24, 2023

Related U.S. Application Data
(60) Provisional application No. 63/070,735, filed on Aug. 26, 2020.
(60) Provisional application No. 63/072,050, filed on Aug. 28, 2020.

(51) Int. Cl. A61B 5/145; A61B 5/1486; A61B 5/00

(52) U.S. Cl. CPC A61B 5/14532; A61B 5/14865; A61B 5/6849; A61B 2562/166; A61B 2560/063; A61B 2562/125; A61B 2560/0285

(57) ABSTRACT

Embodiments of the present disclosure, inter alia, are directed to a one piece continuous glucose monitoring (CGM) device (device or sensor) and a fully disposable, one piece, mounting unit for mounting the CGM device, is provided. The device and the mounting unit, in some embodiments, are pre-assembled in the factory to a one piece, disposable, mounting assembly. The mounting assembly, can be comprised of sealed compartment that includes a portion of the sensor and a non-sealed compartment that includes another portion of the sensor. The mounting assembly can be fully automatic, at a button press the sensor is adhered to the skin, and the sensor probe is inserted into the subcutaneous tissue.

### RELATED APPLICATIONS

This disclosure claims priority to and benefit of U.S. provisional patent application ///no. 63/070,735, filed Aug. 26, 2020, entitled, “Continuous Glucose Sensor and Mounting Assembly,” and U.S. provisional application /no. 63/072,050, filed Aug. 28, 2020, entitled, “Systems, Devices and Methods for Glucose Sensing and associated manufacturing methods.” Each of these disclosures in its entirety is incorporated herein by reference.

### FIELD OF THE DISCLOSURE

Embodiments of the present disclosure are directed to methods, systems, devices, and components thereof, configured for continuous monitoring of glucose in a patient. More particularly:

some embodiments of the present disclosure relate to a disposable glucose sensing device for continuous glucose monitoring; and

some embodiments of the present disclosure relate to a disposable mounting assembly for mounting a glucose sensor on a patient for such monitoring.

### BACKGROUND

Continuous glucose monitoring is important to individuals with diabetes, as they must determine when insulin is needed to reduce glucose levels or when additional glucose is needed to raise the level of glucose. A continuous glucose monitoring device (CGM) usually adheres to a patient's skin and transmits glucose reading to a remote monitoring device. The CGM can be incorporated in a closed loop system (artificial pancreas) in which, an insulin pump automatically administers and adjusts insulin delivery according to CGM transmitted glucose readings (closed loop system).

There exists a need for a fully disposable sensing device for continuous monitoring of glucose. There is also a need for a planar sensor probe having a minimal surface area and maximal electrodes surface area. There is also a need for a mounting device which is easy to use, minimizes pain, and minimizes insertion trauma. There is also a need for a mounting device that is fully automatic in which insertion and retraction of introducer sharp is done by a button press with no patient's intervention in spring pre-loading. There is also a need for a one piece, disposable mounting device that is pre-assembled in the factory and includes the sterilized sensor probe and the non-sterilized components and thus, reducing mounting steps at each device replacement. There is also a need for a one piece disposable mounting device that is cheap for production in which the relatively expensive spring loaded mechanism is not sterilized.

### SUMMARY

Embodiments of the present disclosure, present, among many embodiments, a fully disposable, one piece continuous glucose monitoring (CGM) device (sensing device), which in some embodiments includes a sensor and a sensor probe, and a fully disposable, one piece, mounting assembly/unit for mounting the CGM device (mounting assembly, both mounting unit and mounting assembly used interchangeably). The device and the mounting unit are pre-assembled in the factory to a one piece, disposable, mounting assembly. The mounting assembly is comprised of sealed compartment that includes a portion of the sensor and a non-sealed compartment that includes another portion of the sensor. The mounting assembly can be partially, and preferably, fully automatic, at a button press—the sensor is adhered to the skin, and the sensor probe is inserted into the subcutaneous tissue.

The sensor probe, in some embodiments, includes a first portion which is inserted in the subcutaneous tissue and a second portion adapted to be received in a sensor housing (together the sensor probe, the glucose sensor, and sensor housing comprising a sensing device, as noted above). The sensor housing includes an upper cover and a lower cover, the lower cover including an adhesive, in some embodiments, for adhering the sensor to the skin. Following use, the sensing device (i.e., the sensor housing and sensor probe), in some embodiments, is removed from the body and disposed.

In some embodiments, the sensor comprises an electronic printed circuit board assembly (“PCBA” or “electronics”) and may also include a variety of optional components, such as, for example, a receiver, a transmitter, a processing circuit, a battery, an alarm system, and/or a data storage unit. In some embodiments, the sensor includes a plurality of conductive contacts, e.g., two or more conductive contacts (e.g., conductive springs), that can be configured for coupling to two or more respective contact pads on the sensor probe. The sensor probe, in some embodiments, includes at least one working electrode, a counter electrode, at least two (2) electrical conductors, and at least two (2) contact pads. In some embodiments, the probe is planar and includes two (2) electrodes—a working electrode which can be positioned on one side of the probe, in some embodiments, at the distal end of the probe, and a counter electrode positioned, in some embodiments, on the opposite side. Both electrodes, in some embodiments, are connected with electrical conductors to contact pads that can be positioned on both sides of a contact(s) plate(s). The contact plate, in some embodiments, is perpendicular to the probe (and in some embodiments, can be a part of the probe) and can reside within the sensor housing such that at least one (1) contact pad is facing the PCBA, and another (e.g., the other) contact pad is facing the opposite direction. In some embodiments, the contacts plate is folded on one (1) side at 180 degrees such that both contact pads are facing the PCBA and thus, simplifying electrical connection. In some embodiments, the probe and the contact(s) plate(s) are made from a single matrix sheet (e.g. polyimide) folded such that the probe is configured to be perpendicular to the skin, the contacts plate is perpendicular to the probe (parallel to skin), and both contact pads are facing the PCBA.

According to some embodiments, a mounting unit is provided and configured for mounting the sensing device (e.g., see above embodiments) onto a patient. The mounting unit can be pre-assembled with the sensing device such that the mounting unit and the sensing device (which includes, in some embodiments, the sensor and the sensor probe) are provided in one piece (mounting assembly) that is packed in one box. The mounting unit, in some embodiments, is comprised of two compartments having two pre-assembled housings, a non-sealed, non-sterilized compartment (spring compartment) that includes the insertion and retraction mechanisms and a portion of the sensing device, and a sealed, sterilized compartment (probe compartment) that includes the sensor probe. Both compartments have housings, a spring compartment housing and a probe compartment housing, that are rigidly pre-assembled during manufacture (for example), the spring compartment housing on top of the probe compartment housing. The probe compartment is preferably sealed and sterilized with radiation (e.g., gamma or e-beam). Before device mounting, the patient removes a protecting lid from the bottom side of the sensor probe housing and adheres the mounting assembly to the skin. During device mounting, the sensor and sensor probe are displaced within the sensor probe housing and mounted onto the patient (with the probe being inserted into tissue).

In some embodiments, an introducer for insertion of the sensor probe into the subcutaneous tissue is provided. The introducer, in some embodiments, includes a sharp tip at one end which is configured to penetrate the skin, and an introducer cap, at the other end, which is configured to drive the introducer in one direction during insertion and the opposite direction during retraction. The introducer, in some embodiments, is positioned within the mounting assembly and spans the non-sterilized compartment, the sterilized compartment, and an elastomeric septum (that provides sealing to the sealed, sterilized probe compartment). The introducer can be displaced through the septum in one direction during sensor probe insertion, and then, in an opposite direction during introducer retraction. Following device mounting, the mounting unit can be removed from the body leaving the sensor on the skin and the sensor probe within the subcutaneous tissue.

In some embodiments, after triggering, operation of the mounting unit is automatic. For example, upon pressing an operating button, the mounting unit adheres the sensor to the patient's skin, inserts the sensor probe into the subcutaneous tissue (mounting phase one) and retracts the sensor probe introducer while the sensor probe remains within the body (mounting phase two). After device mounting, the mounting unit can be removed from the body and disposed. In some embodiments, the mounting unit includes a spring loaded driving mechanism for mounting the device onto the patient, and a spring loaded retraction mechanism for removing the introducer while leaving the sensor on the patient. Upon pressing an operating button, a trigger releases a pre-loaded insertion spring that drives the device (including the sensor, sensor probe, and introducer) in a first direction. At the end of movement, a preloaded retraction spring is released, and the introducer is retracted in a second opposite direction such that the introducer sharp is concealed within the mounting unit. In some embodiments, the introducer is a rigid planar structure having a sharp tip and adapted to support the sensor probe during insertion. The introducer and sensor probe can concomitantly be inserted into the subcutaneous tissue; however, in some embodiments, at the end of movement of the introducer at first direction, the sensor probe is further advanced (e.g., for additional 1-3 mm) in the same first direction. Thus, in such embodiments, local trauma within the surrounding tissue can be minimized because the cross profile of the sensor probe is very low (e.g., causes minimal trauma) and the glucose sensing electrode (i.e., the working electrode), that is located at the distal end of the probe, is exposed to a minimal local inflammatory reaction.

An assembly process for the device, according to some embodiments, includes the following steps (which in some embodiments, consecutive steps):

deposition of glucose sensitive layers, electrical conductors, insulators, and contact pads on one or both sides of a matrix sheet;

cutting and/or folding the matrix sheet such that it is configured to include the sensor probe and the contacts plate; and

assembly (and in some embodiments, sterilization) of the probe compartment, and assembly (stacking bottom-up) of the sensor PCBA, sensor housing upper cover, retraction spring, introducer cap and finally, the spring's compartment housing that includes the spring loaded insertion mechanism.

According to some embodiments, a method of mounting the device is provided, which includes the following steps (in some embodiments, consecutive steps): removal of the protecting lid, placement of mounting assembly on the skin, button press, and removal of mounting unit.

In some embodiments, a continuous glucose monitoring system for continuously monitoring glucose levels of a user is provided. The system includes a mounting assembly, and a sensing device, where at least a portion of the sensing device is housed within the mounting assembly prior to mounting the sensing device on the user. It is worth noting, that each of these components are separate embodiments in and to themselves.

The above noted embodiments, including separate embodiments of the mounting assembly and the sensing device, may also include one and/or another (and in some embodiments, a plurality of, a majority of, substantially all of, and in some embodiments, all of) the following additional features, functionality, structure, steps, or clarifications, yielding yet further embodiments (as is clear from the listing below, some of the additional features, functionality, structure, steps, and clarifications—as may be the case—build off of, and/or are based on, previously/earlier recited additional features, functionality, structure, steps, or clarifications):

the sensing device can include at least a transmitter configured to transmit signals corresponding to sensed glucose levels of the user;

a remote display unit configured with at least with a receiver for receiving the sensed glucose readings from the sensing device via the transmitter;

the sensing device can comprise a sensor;

the sensing device can comprise a sensor, and a sensor probe configured for insertion into the subcutaneous tissue of the user;

the sensing device can comprise a sensor configured for adhering to the skin of the user, and a sensor probe configured for insertion into the subcutaneous tissue of the user;
the sensor can include a lower portion (e.g., a lower cover) having an adhesive for removable affixation of the sensor to the skin of the user;
the sensor can include a sensor housing;
the sensor can include a sensor control unit;
the sensor probe can include a first portion configured for insertion into subcutaneous tissue, and a second portion configured for receiving by the sensor housing; and/or
the sensor probe can include a first portion configured for insertion into subcutaneous tissue, and a second portion configured for receiving by the sensor housing;

the sensor housing can comprise an upper cover and a lower cover; where the lower cover can include an adhesive for adhering the sensor to the skin of the patient;

the sensor can include a printed circuit board assembly (PCBA);

at least one of the sensor housing and the PCBA can also include at least one, a plurality of, a majority of, substantially all of, or all of, a receiver, a transmitter, a processing circuit, a battery, an alarm system, and data storage means, where the receiver and the transmitter can together comprise a transceiver;

the sensor can include at least one, and preferably, a plurality of conductive contacts configured for coupling to one or more respective contact pads of the sensor probe;

the sensor probe can include:
at least one of: at least one working electrode, a counter electrode, and at least two (2) electrical conductors, and at least two (2) contact pads; or
includes at least one working electrode, a counter electrode, and at least two (2) electrical conductors, and at least two (2) contact pads;

the sensor probe can be configured with a planar shape, and includes at least two (2) electrodes, where the at least two (2) electrodes comprises at least a working electrode;

the working electrode can be configured for positioning on at least one of a first side of the sensor probe and at a distal end thereof;

the at least two (2) electrodes can comprise a counter electrode;

the at least two (2) electrodes can comprise at least a working electrode and a counter electrode, where the working electrode is configured for positioning on at least one of a first side of the sensor probe and at a distal end thereof, and the counter electrode is configured for arrangement on a side of the sensor probe opposite to the side where the working electrode is positioned on the sensor probe;

each electrode can be connected via electrical conductors to a respective contact pad;

one or more contact pads can be configured:
for positioning on at least one side of a contacts plate, the contacts plate optionally included with the sensor probe; or
for positioning on each side of a contacts plate, the contacts plate optionally included with the sensor probe;

the contacts plate can be:
positioned approximately perpendicular to the sensor probe; and/or
positioned within the sensor housing;

upon the contacts plate being positioned within the sensor housing (see above), at least one (1) contact pad can be facing the PCBA, and optionally, another (e.g., the other) contact pad can be facing an opposite direction;

the contacts plate can be folded on a first side at 180 degrees, such that each contact pad is facing the PCBA (see above);

the sensor probe and the contacts plates can be constructed from a matrix sheet configured such that the sensor probe is perpendicular to the skin, the contacts plate is perpendicular to the sensor probe (parallel to skin), and both contact pads are facing the PCBA;

the matrix sheet can comprise a single matrix sheet;

the mounting assembly can be configured for mounting the sensing device onto a patient;

the mounting assembly can be assembled with the sensing device during manufacture, such that it is ready to mount the sensing device onto a patient;

the mounting assembly can comprise a first, non-sealed, non-sterilized compartment, and a second sealed, sterilized compartment (in some embodiments, both compartments can be sterilized);

at least one of the first and second compartments can each include a housing;

the first compartment can be arranged immediately adjacent the second compartment;

the first compartment can include at least one of an insertion means and a retraction means;

the first compartment can include an insertion means and a retraction means;

the second compartment can include at least one of the sensor probe and a portion of the sensor;

the second compartment can include the sensor probe and a portion of the sensor, and optionally an elastomeric septum configured to sealing the second compartment;

the second compartment can include a protection lid;

a side of the second compartment from which the protection lid is removed can be configured for placement adjacent to the skin of the patient;

upon mounting the sensing device, at least one of the sensor and sensor probe can be displaced within the second compartment and mounted onto the patient;

upon mounting the sensing device, the sensor and sensor probe can be displaced within the second compartment and mounted onto the patient;

a sensor-probe introducer (“introducer”) configured to insert the sensor probe into the subcutaneous tissue of the patient;

the introducer can comprise at least one of, a plurality of, a majority of, substantially all of, or preferably all of:
a sharp tip at a first end configured to penetrate the skin of the patient, and
an introducer cap, at a second end configured to drive the introducer in a first direction during insertion and a second direction opposite the first direction during retraction;

and

the introducer:
can be positioned within the mounting assembly;
can be positioned within the mounting assembly and spans the first compartment, the second compartment, and the elastomeric septum;
can be configured for displacement through the septum in a first direction during sensor probe insertion, and in a second direction opposite to the first direction during introducer retraction;
can comprise a rigid planar structure including a/the sharp tip, and is adapted to support the sensor probe during insertion;
with the sensor probe, can concomitantly be inserted into the subcutaneous tissue;
can be planar in cross-section;
can be not oval or round in cross-section;
can include a u-shaped cross-section; and/or
can include at least one and preferably two shoulders configured to at least one of secure the sensor probe in insertion and allow introducer retraction, while leaving the sensor probe in the body;

the mounting assembly can be configured to automatically operate, including automatically adhering the sensor to the patient's skin, inserting the sensor probe into the subcutaneous tissue, and retracting the sensor probe introducer;

a trigger configured to initiate the automatic operation of the mounting assembly to mount the sensing device on the user;

the trigger can include an operating button or an operating switch configured to receive user or operator input;

the insertion means can comprise a spring loaded driving mechanism configured to mount the sensing device onto the patient;

the retraction means can comprise a spring loaded retraction mechanism for removing the introducer while leaving the sensing device on the patient;

upon pressing a/the operating button, the trigger can be configured to release the insertion spring of the insertion means that drives the sensing device, including the sensor, the sensor probe, and optionally the introducer, in a first direction, thereafter, the retraction spring can be configured to release and the introducer can be retracted in a second opposite direction such that the sharp end of the introducer can be at least substantially concealed within the mounting unit;

at the end of insertion, the sensor probe can be further advanced in the first direction (e.g., 1-3 mm);

both the first and the compartments can be sterilized compartments;

and

the second compartment (i.e., probe compartment) can be sterilized first with an e-beam and/or gamma radiation, followed by the first compartment (i.e., spring compartment) which can be sterilized with a gas (e.g., ethylene oxide).

In some embodiments, a glucose monitoring sensing device mounting assembly according to any of the above noted embodiments (and any other embodiments of this disclosure) is provided.

In some embodiments, a glucose monitoring sensing device according to any of the above-noted embodiments (and any other embodiments of this disclosure) is provided.

In some embodiments, an assembly method for assembling a mounting assembly and a sensing device for a continuous glucose monitoring system is provided and includes assembling a/the sensing device and assembling a/the mounting assembly (each according to any of the disclosed embodiments). For assembling the sensing device, the method includes at least one of, and preferably a plurality of, and most preferably all of:

depositing glucose sensitive layers, electrical conductors, insulators, and contact pads on one or both sides of a matrix sheet, such depositing includes forming one or more electrodes on the matrix sheet;

cutting and/or folding the matrix sheet such that it is configured to include the sensor probe and the contacts plate; and

assembling of the sensor contacts plate, sensor printed-circuit-board-assembly (PCBA), and sensor housing as to form a sensor

For assembling of the mounting assembly, the method includes at least one of, and preferable a plurality of, and most preferably all of:

assembling at least one of an insertion spring and a retraction spring into a first compartment of the mounting assembly;

assembling and sterilizing a second compartment of the mounting assembly; and

placing at least a portion of the sensing device in the second compartment.

Such method embodiments, including separate embodiments of the mounting assembly and the sensing device, may also include one and/or another (and in some embodiments, a plurality of, substantially all of, and in some embodiments, all of) the following additional features, functionality, structure, steps, or clarifications, yielding yet further embodiments (as is clear from the listing, some of the additional features, functionality, structure, steps, and clarifications—as may be the case—build off of, and/or are based on, previously/earlier recited additional features, functionality, structure, steps, or clarifications):

both the first and the second compartments can be sterilized;

the second compartment is sterilized first with an e-beam and/or gamma radiation, followed by the first compartment which is sterilized with a gas (e.g., ethylene oxide);

at least one of the probe and contacts plate are formed on/from the matrix sheet;

the sensor probe and/or the contacts plate can be formed on/from the matrix sheet;

at least one of the sensor probe and the contacts plate can be formed on/from the matrix sheet via cutting and/or folding so as to form an/the appropriate spatial configuration;

at least one of the one or more electrodes, one or more conductors, and one or more contact pads, can be positioned on at least one side of the matrix sheet;

at least one of the one or more electrodes, one or more conductors, and one or more contact pads, can be positioned on both sides of one matrix sheet;

and

the folding of contacts plate can be configured to arrange the contact pads to face the same direction, where the same direction can be towards the PCBA.

These and other embodiments, advantages, and objects thereof, of the various inventions of the present disclosure are even more evident given the detailed description of at least some of the embodiments which follow, as well as the figures that form part of this disclosure (a brief description of which is outlined below).

### BRIEF DESCRIPTION OF THE DRAWINGS

FIG. 1 shows a scheme of the CGM system that includes a mounting assembly and the display unit, according to some embodiments.

FIGS. 2A-C show cross-sectional view (2A), spatial/perspective (2B), and oblique cross section (2C) views of a mounting assembly, according to some embodiments.

FIGS. 3A-B show exploded (3A) and cross-section (3B) views of a probe compartment, according to some embodiments.

FIG. 4 shows a cross-sectional view of a mounting assembly after removal of a protecting lid, according to some embodiments.

FIG. 5 shows a cross-sectional view of a mounting assembly at the end of a first mounting phase, according to some embodiments.

FIG. 6 shows a cross-sectional view of a mounting assembly at the end of a second mounting phase, according to some embodiments.

FIGS. 7A-B shows a spatial view (7A) and a cross-sectional view (7B) of a mounting assembly after removal from a sensor, according to some embodiments.

FIGS. 8A-B show a spatial view (8A) and a cross-sectional view (8B) of a sensor, according to some embodiments.

FIG. 8C shows a cross section view of a sensor on a patient, according to some embodiments.

FIG. 9A shows a cross section view of a spring release mechanism, according to some embodiments.

FIGS. 9B and 9C are magnified views of a release mechanism of an insertion spring (9B) and a retraction spring (9C), respectively, according to some embodiments.

FIG. 10 shows an exploded view of parts of a sensor, according to some embodiments.

FIG. 11 shows a sensor according to some embodiments.

FIG. 11A shows a top level view of a sensor 50, according to some embodiments.

FIG. 11B shows a spatial, exploded view of a sensor, according to some embodiments.

FIG. 11C shows a cross-sectional view of a septum and an introducer configured to cross the septum, according to some embodiments.

FIG. 12 shows parts of a sensor, according to some embodiments.

FIG. 12A shows a spatial view of a sensor printed circuit board (PCBA), a sensor base plate, and an introducer, according to some embodiments.

FIG. 12B shows a spatial view of an introducer, a contacts plate, and a conductive spring, according to some embodiments.

FIG. 12C shows an exploded view of an introducer, a sensor probe, a contacts plate, conductive springs, and conductive spring holders, according to some embodiments.

FIGS. 13A-A1 show a sensor probe and a contacts plate after (13A1) and before (13A) folding of a matrix sheet, according to some embodiments.

FIGS. 13B-B1 show spatial views of mirror images of a sensor probe and a contacts plate, electrodes, conductors, and contact pads, according to some embodiments.

FIG. 14 show spatial views of an introducer, a probe and a contacts plate, according to some embodiments.

FIG. 14A shows an exploded view of an introducer and a probe before sensor probe insertion, according to some embodiments.

FIGS. 14B-C show an introducer and a sensor probe before insertion, with FIG. 14B showing a magnified view of an introducer tip and a probe tip before sensor probe insertion, and FIG. 14C showing a magnified view of the introducer tip and the probe tip after probe insertion (before introducer retraction), according to some embodiments.

FIGS. 15A1-3 show a side view (15A1), a top view (15A2), and a cross-sectional view (15A3) of an introducer tip, according to some embodiments.

FIGS. 15B1-2 show a side view (15B1) and a top view (15B2) of an introducer tip and a sensor probe tip, according to some embodiments.

FIGS. 15C and 15D1-2 show top views of some preferred embodiments of sensor probe tip 65, according to some embodiments.

FIGS. 16A-C and 16A1-C1 show spatial and magnified views, respectively, of a sensor, a sensor probe, an introducer tip, and a probe tip, during first and second mounting phases (i.e., insertion of introducer and sensor probe, and retraction of introducer, respectively), according to some embodiments.

FIGS. 16A-A1 show a sensor probe coupled with an introducer, according to some embodiments.

FIGS. 16B-B1 show a probe tip protruding from an introducer tip, according to some embodiments.

FIGS. 16C-C1 show a sensor and a probe after removal of an introducer, according to some embodiments.

FIGS. 17 and 18 show an assembly process of a mounting assembly, according to some embodiments.

FIGS. 17A-B show exploded view (17A) and oblique cross-sectional view (17B) of a sterilized, sealed compartment (a second/probe compartment), according to some embodiments.

FIGS. 18A1-F1 and 18A2-F2 show spatial views (18A1-F1) and oblique cross-sectional views (18A2-F2), respectively, of consecutive assembly stages of a mounting assembly, according to some embodiments.

### DETAILED DESCRIPTION

The following reference numbers and associated terms are used to describe various structure, according to at least some embodiments of the present disclosure.
1 Mounting assembly
2 Display unit
5 matrix sheet (after folding forms the sensor probe and contacts plat)
6 Probe compartment (in some embodiments, referred to as a/the second compartment)
7 First electrode (working)
8 Second electrode (counter)
9 Spring compartment
10 Mounting unit
11 Operating button
12 Trigger
13 Sleeve
14 Hammer
141 Hammer introducer pulling lever
142 Hammer sensor pushing lever
15 Spring compartment base
155 Retraction spring latch
16 Probe compartment housing
161 Probe compartment housing worm
162 Probe compartment seal
17 Protecting lid
171 Protecting lid supporting ribs
172 Protecting lid thread
173 Protecting lid base
174 Protecting lid seal
18 Trigger latches
19 Spring compartment housing
20 Introducer
21 Introducer cap
211 Introducer cap snap
212 Introducer cap snap holder
213 Introducer cap knob
22 Introducer tip
23 Introducer probe holder
31 Insertion spring
32 Retraction spring
50 Sensor
51 Sensor housing upper cover
52 Sensor septum
53 Sensor printed circuit board assembly (PCBA)
533 print opening
54 Sensor base plate
541 Base plate opening
542 Conductive spring
543 Conductive spring holder
544 Base plate battery groove
546 Base plate seal
55 Sensor housing lower cover
551 Lower cover cushion pad
56 Sensor adhesive
60 Sensor probe
65 Sensor probe tip
61 Contacts plate
611 Contacts plate first side
7 First electrode (working)
71 Conductor first electrode (working)
72 Contact pad first electrode (working)
612 Contacts plate second side
8 Second electrode (counter)
81 Conductor second side (counter)
82 Contact pad second electrode (counter)
613 Contacts plate fold
70 Subcutaneous tissue
80 Skin

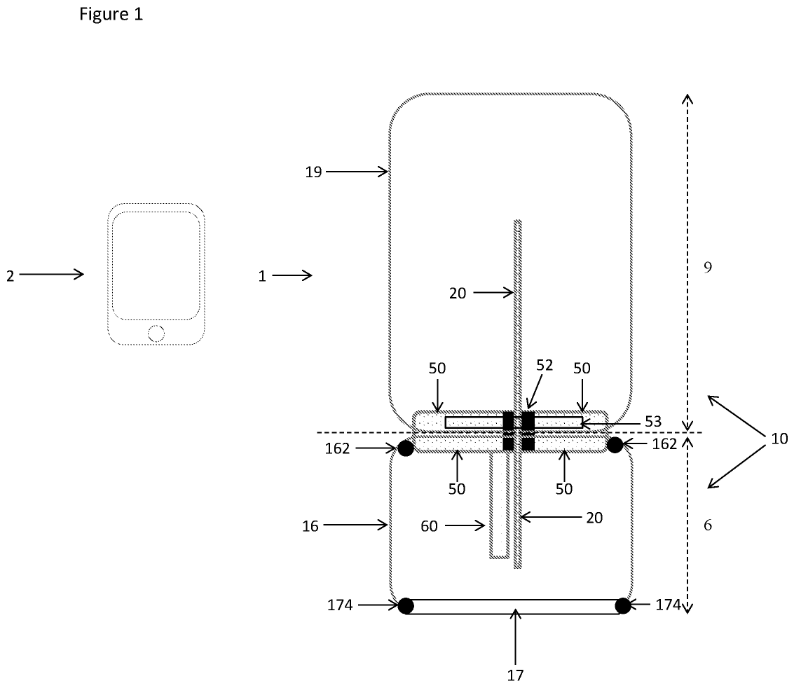

FIG. 1 shows a scheme of a continuous-glucose-monitoring (CGM) system, according to some embodiments, that includes an implantable subcutaneous glucose sensitive probe for detecting glucose levels in the interstitial fluid of a patient. The CGM system, in some embodiments, includes a disposable mounting assembly 1 and a durable remote display unit 2. Remote display unit 2, in some embodiments, has a receiver for receiving glucose readings from a skin adhered glucose sensor (“sensor” or “glucose sensor” 50), and may also include a screen for presenting the received glucose readings. Mounting assembly 1, according to some embodiments, is a one-piece, disposable unit that includes sensor 50 and a mounting unit 10 for mounting sensor 50 onto a patient. Mounting unit 10 and sensor 50, in some embodiments, are pre-assembled during manufacture, and thus, are provided in one single disposable unit—mounting assembly 1. Before sensor mounting (on the body), sensor 50, in some embodiments, at least a portion of the sensor is concealed, and in some embodiments, substantially all or all of the sensor is concealed, within the mounting unit 10.

Mounting assembly 1, in some embodiments, is comprised of two (2) compartments: a non-sterilized, non-sealed compartment, which may also be referred to as a first or spring compartment 9, and a second, sterilized, sealed compartment, which may also be referred to as a probe compartment 6. The sensor, in some embodiments, comprises at least two (2) components that can be pre-assembled during manufacture: a sensor control unit, which in some embodiments, includes a housing adapted for placement on the skin, and a sensor probe 60 that has a first portion configured for insertion in the subcutaneous tissue and a second portion configured for being received in a sensor housing. Sensor probe 60, in some embodiments, includes a glucose sensitive enzyme configured for detecting glucose levels in the interstitial fluid within the subcutaneous tissue. Sensor 50, in some embodiments, is positioned within mounting assembly 10 such that at least a first portion of sensor 50 can be received in the sealed, sterilized compartment (second/probe compartment 6), and at least another portion (e.g., second portion) of sensor 50, which includes a PCBA 53, which is adopted to be received in the non-sealed, non-sterilized compartment (spring compartment 9).

In some embodiments, both compartments of mounting assembly 1 are sterilized. In this configuration, second/probe compartment 6 is sterilized with, for example, an e-beam or gamma radiation, and spring compartment 9 is sterilized with a gas (e.g., ethylene oxide). The portion of sensor 50 which includes PCBA 53, in some embodiments, is configured to be received in a sterilized compartment (spring compartment 9).

In some embodiments, an introducer 20 is included, which is configured for inserting sensor probe 60 into subcutaneous tissue. Introducer 20, in such embodiments, may be positioned within mounting assembly 1, and can span non-sterilized spring compartment 9, sterilized probe compartment 6, and an elastomeric septum 52. Probe compartment 6 can include a housing 16, a removable lid 17, a portion of sensor 50, and a portion of introducer 20. Sealing can be provided by a protecting lid seal 174, probe compartment seal 162, and septum 52.

The spring compartment, in some embodiments, includes a housing 19, a portion of introducer 20, and spring mechanisms (not shown) for driving introducer 20 in a first direction (e.g., probe 60 insertion), and a second direction, opposite to the first direction (e.g., introducer 20 retraction). In some embodiments, before use, a patient removes protecting lid 17 providing a free forward movement (e.g., in FIG. 1—downward movement) of sensor 50 and sensor probe 60 within probe compartment housing 16.

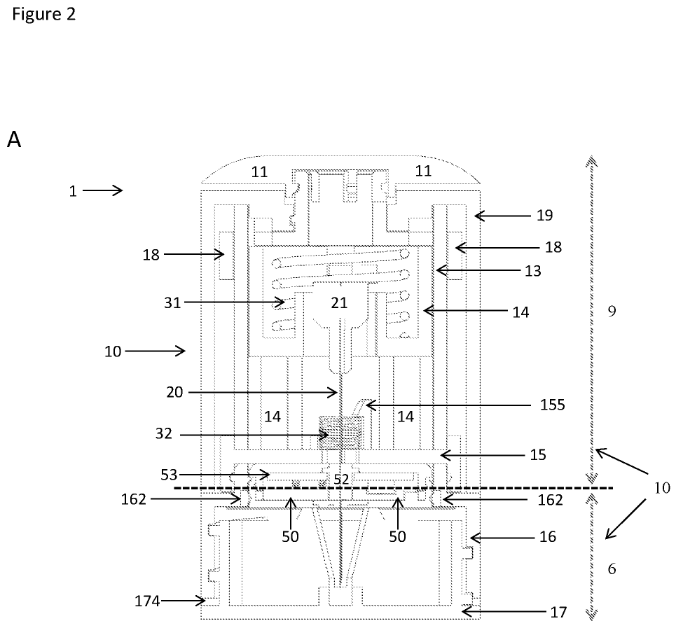

FIGS. 2A-C show cross-sectional (2A), spatial (2B), and oblique cross-sectional (2C) views of mounting assembly 1 that, in some embodiments, is comprised of mounting unit 10 and sensor 50. FIG. 2A shows mounting unit 10 that can be comprised of probe compartment 6 and spring compartment 9. Probe compartment 6, spring compartment 9, and sensor 50, in some embodiments, can be pre-assembled during manufacture, forming mounting assembly 1. The probe compartment can include probe compartment housing 16, removable protecting lid 17, a portion of sensor 50, sensor probe (not shown), and at least a portion of introducer 20. Sealing of probe compartment 6 can be provided by protecting lid seal 174, probe compartment seal 162, and septum 52. Spring compartment 9 can include a spring compartment housing 19, a portion of introducer 20 including introducer cap 21, and one or more spring mechanisms (e.g., insertion and retraction spring loaded mechanisms).

The spring mechanisms, in some embodiments, include at least a plurality of (and in some embodiments, all of) an operating button 11, a trigger 12, trigger latches 18, a sleeve 13, hammer 14, insertion spring 31, retraction spring 32, and retraction spring latch 155. Accordingly, following a press of operating button 11, trigger 12 releases hammer 14, insertion spring 31 drives hammer 14 in a first direction (e.g., forward) which drives introducer cap 21, introducer 20, and sensor 50 in the same direction. When introducer cap 21 reaches the end of the forward movement (e.g., end of a first mounting phase), in some embodiments, it releases retraction spring latch 155 and retraction spring 32 drives introducer cap 21 and introducer 20 in a second direction, opposite to the first direction (e.g., backward) direction.

FIG. 2B shows a spatial view of one-piece mounting unit 10, including probe compartment 6, probe compartment housing 16, spring compartment 9, spring compartment housing 19, and operating button 11. FIG. 2C shows an oblique cross-sectional view of mounting assembly 1, according to some embodiments, that includes probe compartment housing 16, spring compartment housing 19, sensor 50, sensor probe 60, and introducer 20. Removable protecting lid 17 can include a protecting lid base 173, and can be connected to probe compartment housing 16 with a protecting lid thread 172. In some embodiments, protecting lid supporting ribs 171 provide support to the probe compartment and sensor probe protection. Introducer 20 can be connected at one end (e.g., proximal end) to an introducer cap 21, that, in some embodiments, is comprised of introducer cap snap 211, introducer cap snap holder 212, and introducer knob 213.

In some embodiments, the insertion mechanism includes operating button 11, trigger 12, sleeve 13, hammer 14, and insertion spring 31. The hammer 14 can include the introducer pulling lever 141, and sensor pushing lever 142 (e.g., three (3) levers). In some embodiments, the release mechanism includes retraction spring 32 and retraction spring latch 155. In some embodiments, before use, the protecting lid is removed by patient, and upon pressing of operating button 11, trigger 12 releases the hammer 14 and insertion spring 31 drives hammer 14 in a (first) forward direction within sleeve 13. In some embodiments, hammer introducer pulling lever 141, that is connected to introducer cap snap 211, drives introducer 20 in the (first) forward direction, and concomitantly (in some embodiments), the hammer sensor pushing levers 142 drive sensor 50 and sensor probe 60 also in the (first) forward direction. At the end of forward movement of hammer 14 (end of the first mounting phase), introducer cap knob 213 releases retraction spring 32 which drives introducer cap 21 in an opposite (second), e.g., backward, direction.

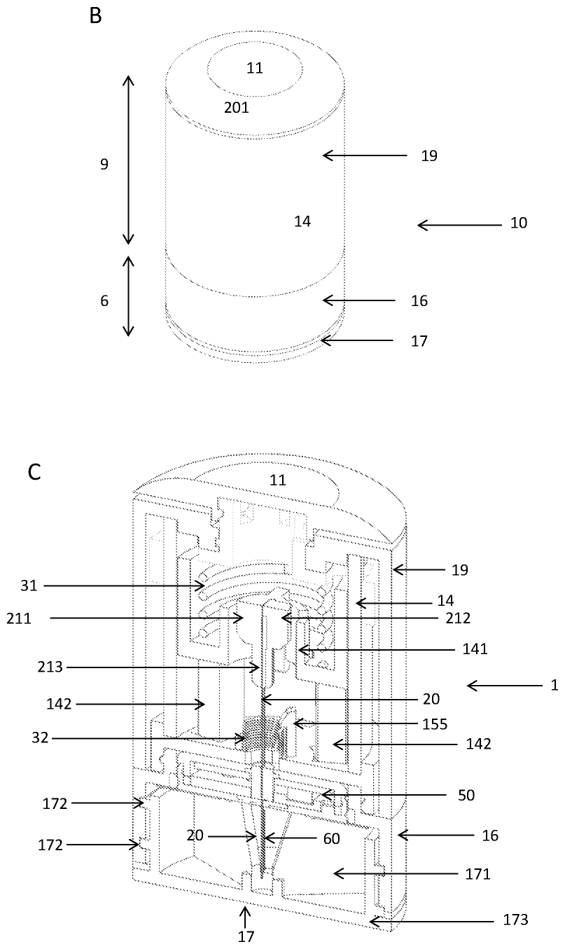

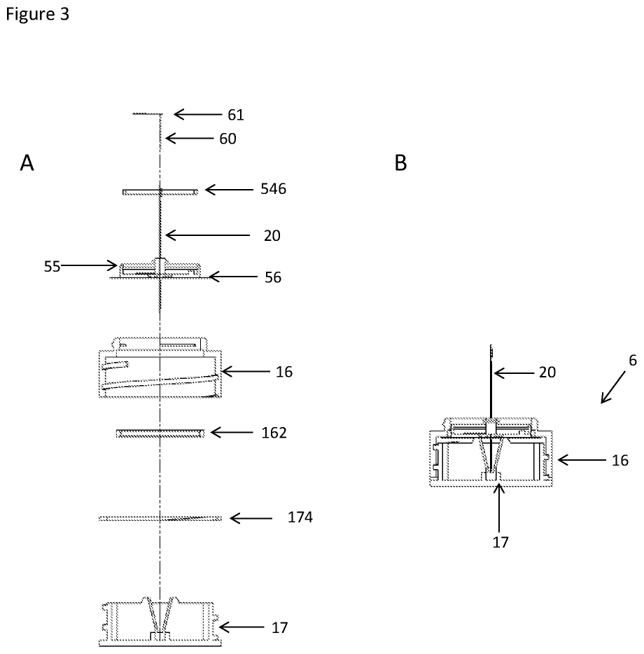

FIGS. 3A-B show exploded (3A) and cross-sectional (3B) views of the sealed, sterilized, probe compartment 6, according to some embodiments. FIG. 3A shows parts of probe compartment 6 (stacked, e.g., from the bottom up): protecting removable lid 17, protecting lid seal 174, probe compartment seal 162, probe compartment housing 16, sensor housing lower cover 55 and adhesive 56, introducer 20, sensor base plate 54 (not shown), base plate seal 546, sensor probe 60, and contacts plate 61. Protecting lid seal 174 and probe compartment seal 174 can be included so as to provide sealing to the probe compartment 6 before mounting the sensor 50 to a patient's body. Sensor base plate seal 546 can be included to provide sealing to the sensor after sensor mounting while the sensor is adhered to the patient's skin. FIG. 3B shows a cross-sectional view of an assembled probe compartment 6: sensor probe housing 16, introducer 20, and protective lid 17 (shown).

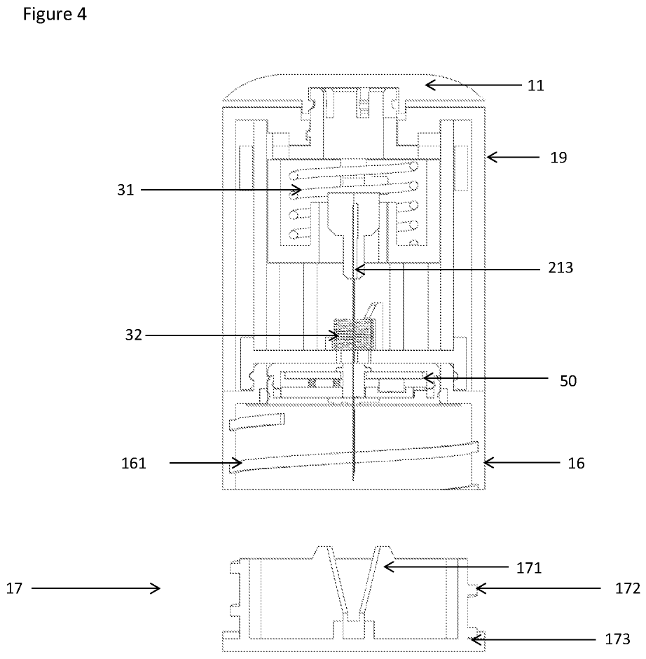

FIG. 4 shows a cross-sectional view of mounting assembly 1 after removal of protecting lid 17 (before sensor mounting), according to some embodiments. Accordingly, mounting assembly 1 can include spring compartment housing 19, sensor probe housing 16, insertion spring 31, retraction spring 32, and introducer cap knob 213. Protecting lid 17, can include protecting lid base 173, protecting lid thread 172, and protecting lid ribs 171. Before sensor 50 mounting, the protecting lid 17, in some embodiments, is removed by the patient by unscrewing the thread 172 from the probe compartment housing worm 161.

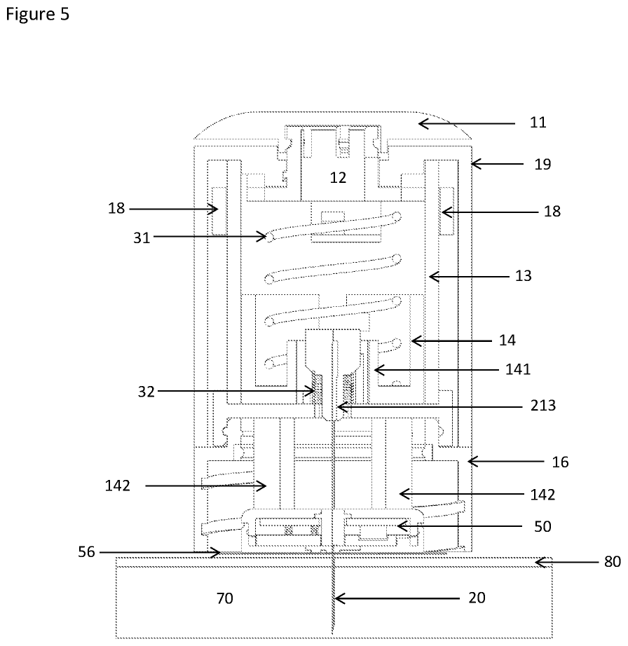

FIG. 5 shows a cross-sectional view of mounting assembly 1 at the end of hammer 14 forward (first direction) movement (end of the first mounting phase). Accordingly, sensor 50 is adhered to the skin 80 with adhesive 56 and introducer 20 and sensor probe (not shown) are inserted within the subcutaneous tissue 70. Mounting assembly 1, in such embodiments, can include a plurality of, and in some embodiments, all of, spring compartment housing 19, probe compartment housing 16, operating button 11, trigger 12, trigger latches 18, sleeve 13, hammer 14, hammer sensor pushing levers 142, insertion spring 31, retraction spring 32, introducer 20, and introducer knob 213. Following the pressing of operating button 11, trigger 12 releases the hammer 14 and insertion spring 31 drives hammer 14 in a forward (first) direction within the sleeve 13. In some embodiments, at the end of forward movement of hammer 14, introducer knob 213 is positioned within the loaded retraction spring 32, sensor 50 is adhered to the skin 80, and introducer 20 and sensor probe 60 are inserted within the subcutaneous tissue 70.

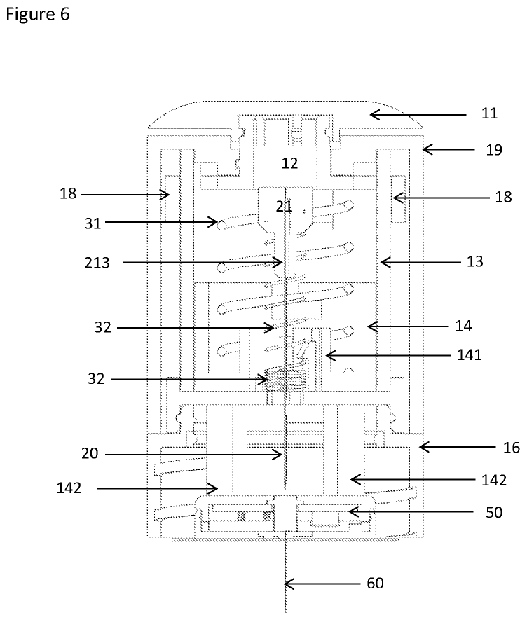

FIG. 6 shows a cross-sectional view of the mounting assembly at the end of introducer 20 retraction (end of a second mounting phase). The mounting assembly, in some embodiments, can include a plurality of, and in some embodiments, all of, probe compartment housing 16, spring compartment housing 19, operating button 11, trigger 12, sleeve 13, hammer 14, hammer introducer pulling lever 141, hammer sensor pushing levers 142, insertion spring 31, retraction spring 32, introducer 20, introducer cap 21, and introducer knob 213. Accordingly, in some embodiments, sensor 50 is adhered to skin (not shown) and sensor probe 60 is inserted within the subcutaneous tissue (not shown). Following introducer 20 insertion, (i.e., the end of forward/first movement of hammer 14), retraction spring 32 is released and the introducer cap 21, introducer knob 213, and introducer 20 are driving at the opposite direction (backward/second-direction movement). At the end of the second mounting phase, introducer 20 is concealed within mounting assembly 1.

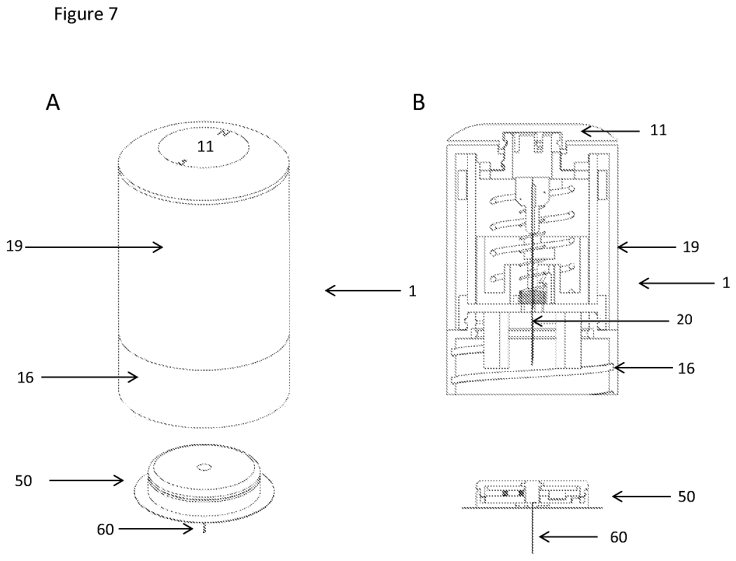

FIGS. 7A-B show spatial (7A) and cross-sectional (7B) views of mounting assembly 1 after removal from the body, sensor 50, in some embodiments, is mounted on the skin (not shown) and sensor probe 60 is inserted in the subcutaneous tissue (not shown). Mounting assembly 1 includes probe compartment housing 16, spring compartment housing 19, operating button 11, and introducer 20.

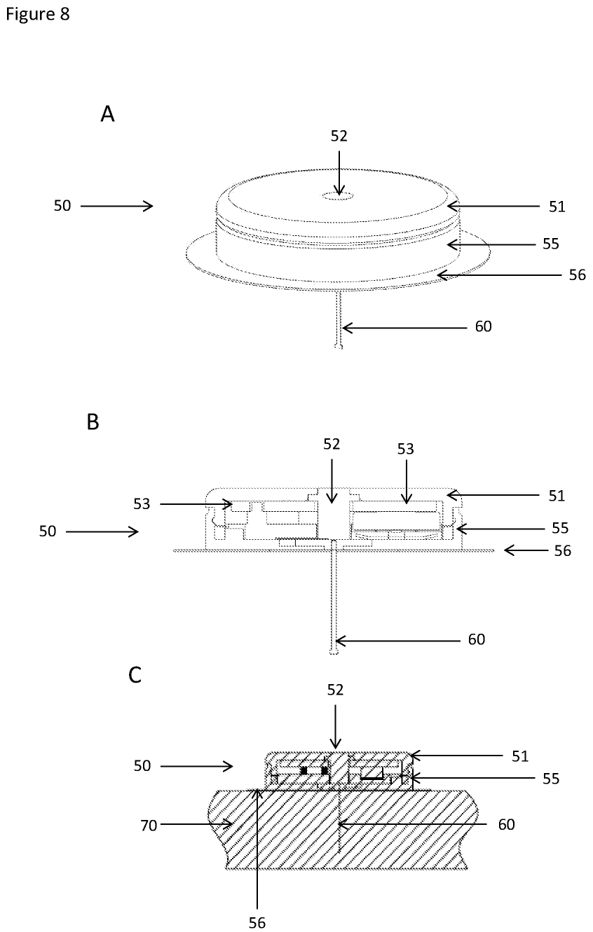

FIGS. 8A-B show spatial (8A) and cross-sectional (8B) views of sensor 50, according to some embodiments. FIG. 8A shows sensor 50 and sensor probe 60, with sensor 50 including, in some embodiments, sensor housing upper cover 51, sensor housing lower cover 55, septum 52, and adhesive 56. FIG. 8B shows the sensor housing upper cover 51, sensor housing lower cover 55, PCBA 53, septum 52, and adhesive 56. The sensor housing lower cover 55, sensor base plate 54 (not shown), septum 52, and adhesive 56 can be configured to be received in the sterilized compartment (e.g., probe compartment 6), and PCBA 53 and sensor housing upper cover 51 are configured to be received in the non-sterilized compartment (e.g., spring compartment 9). FIG. 8C shows a cross section view of the sensor 50 on a patient skin, the sensor probe 60 is inserted within the subcutaneous tissue. The sensor 50 includes the sensor housing upper cover 51, sensor housing lower cover 55, septum 52, and adhesive 56.

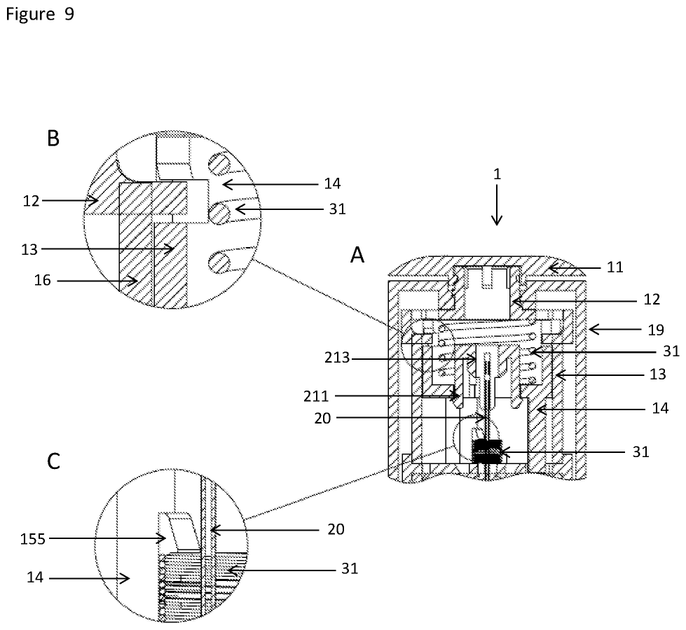

FIG. 9 shows the spring release mechanisms, according to some embodiments. FIG. 9A shows a cross-sectional view (cross-section plan is 45° rotated vs. FIG. 2 cross section plan) of the spring release mechanisms. FIGS. 9B and 9C are magnified views of the release mechanisms of insertion spring 31 (9B) and retraction spring 32 (9C), respectively. FIG. 9A shows mounting assembly 1 that includes at least a plurality of, and in some embodiments, all of: spring compartment housing 19, operating button 11, trigger 12, sleeve 13, hammer 14, insertion spring 31, retraction spring 32, introducer 20, introducer cap snap 211, and introducer cap knob 213. During insertion, and in some embodiments, hammer 14 is displaced in a forward (e.g., first) direction by loaded spring 31. The introducer cap snap 211 is displaced by hammer 14 in the same (forward/first) direction and concomitantly drives introducer 20 and introducer cap knob 213 in the same (forward/first) direction. FIG. 9B shows the release mechanism of insertion spring 31, according to some embodiments, where upon pressing operating button 11, the trigger 12 is displaced in the same (forward/first) direction and rotates (in some embodiments, slightly, e.g., less than one rotation) such that hammer 14 is released and displaced (e.g., forward) within sleeve 13 by loaded spring 31.

FIG. 9C shows the retraction mechanism of retraction spring 32, according to some embodiments. Here, retraction spring latch 155 is laterally displaced by the introducer cap knob 213, and releases the retraction spring 32. Retraction spring 32 is then displaces the introducer 20 in a second/backward direction opposite to the first/forward direction.

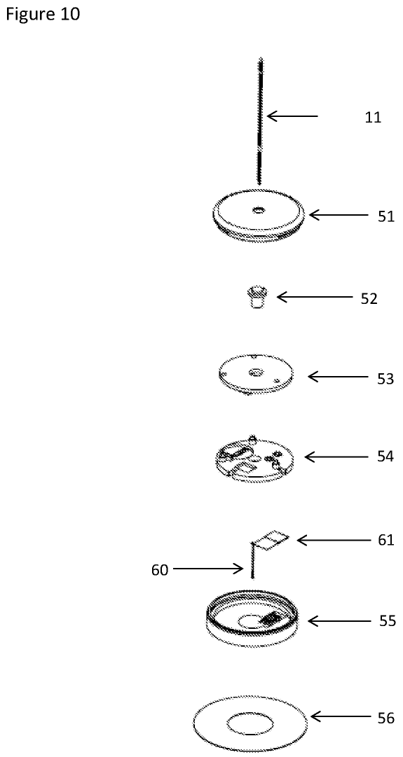

FIG. 10 shows an exploded view of parts/components of sensor 50 and sensor probe 60, according to some embodiments. Sensor 50 and sensor probe 60 can include (in a stacked arrangement, e.g., stacking bottom-up) at least a plurality of, and in some embodiments, all of: adhesive 56, sensor housing lower cover 55, sensor probe 60, contacts plate 61, sensor base plate 54, PCBA 53, septum 52, and sensor housing upper cover 51. The adhesive 56, sensor housing lower cover 55, sensor base plate 54, sensor probe 60, contacts plate 61, septum 52 and a portion of introducer 20 are sterilized (according to some embodiments); PCBA 53 and sensor housing upper cover 51 are not sterilized (according to some embodiments).

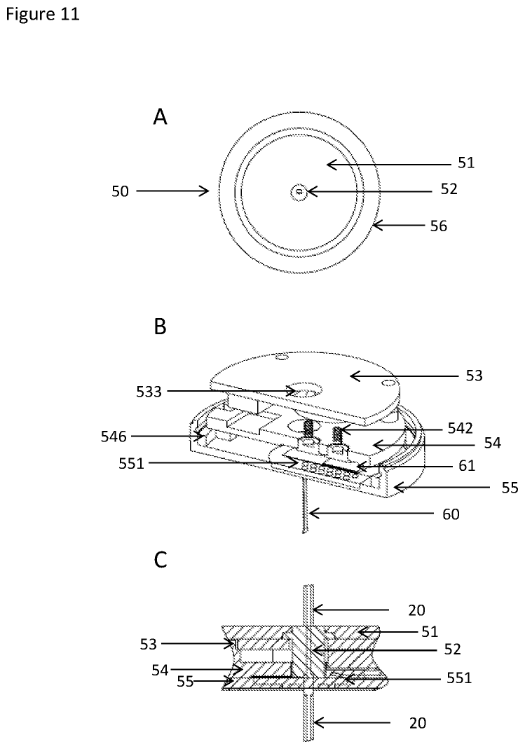

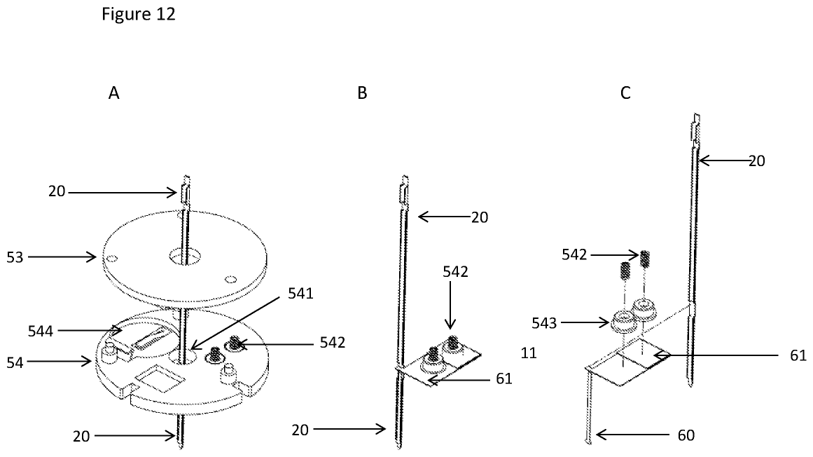

FIG. 11 shows sensor 50 components, according to some embodiments. FIG. 11A shows a top level view of sensor 50, according to some embodiments, that includes adhesive 56, septum 52, and sensor housing upper cover 51. FIG. 11B shows a spatial, exploded view of sensor 50 and sensor probe 60. Sensor 50 can include a plurality of, and in some embodiments, all of sensor housing lower cover 55, lower cover cushion pad 551, contacts plate 61, base plate seal 546, sensor base plate 54, conductive springs 542, PCBA 53, and PCBA opening 533. The base plate seal 546, in some embodiments, provides sealing (e.g., water resistance) for sensor 50 after connection of sensor housing lower cover 55 and sensor housing upper cover 51. The lower cover cushion pad 551, in some embodiments, can provide tight/rigid connection between contacts pad (shown in FIGS. 12-14) that are located on contacts plate 61 and the conductive springs 542. In some embodiments, during sensor operation, electrical current generated on sensor probe 60 is conducted to contacts plate 61 and through conductive springs 542 to the PCBA 53. FIG. 11C shows a cross-sectional view of a portion of sensor 50 that includes, according to some embodiments, sensor housing lower cover 55, sensor base plate 54, PCBA 53, and sensor housing upper cover 51. In some embodiments, introducer 20 crosses (e.g., through/in) septum 52.

FIG. 12 shows the sensor components/parts, according to some embodiments. FIG. 12A shows a spatial view of the sensor electronic print (PCBA) 53, sensor base plate 54, and introducer 20. Sensor base plate 54 can include at least one of, and preferably both of, a recess 544 for housing the battery (not shown), base plate opening 541, and openings for the conductive springs 542. Introducer 20 can span the sensor through openings in PCBA 53 and sensor base plate 54 (septum 52 that crosses these openings is not shown). FIG. 12B shows a spatial view of introducer 20, contacts plate 61, and conductive springs 542, according to some embodiments. FIG. 12C shows an exploded view of introducer 20, sensor probe 60, contacts plate 61, conductive springs 542, and conductive spring holders 543 (sensor base plate 54 and PCBA 53 removed), according to some embodiments.

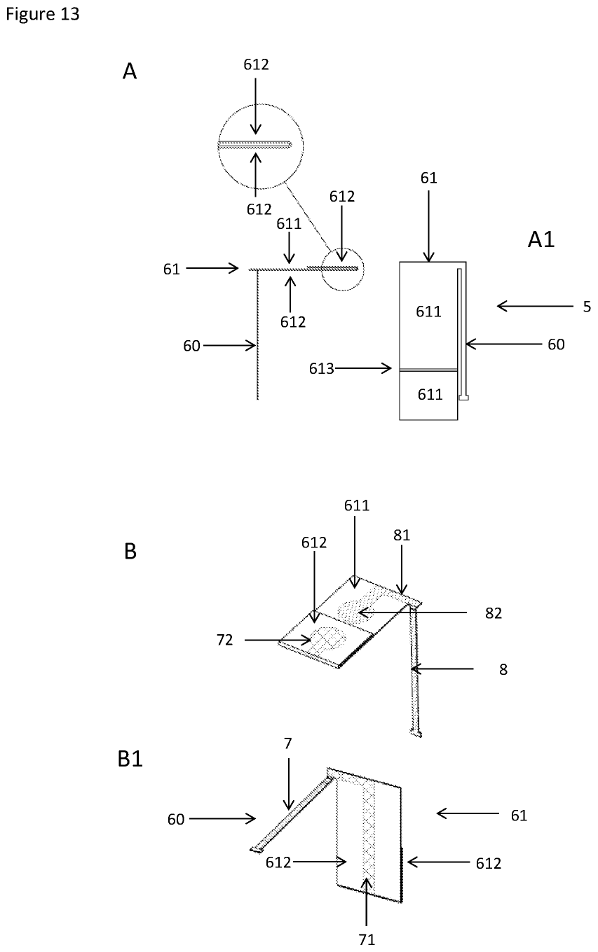

FIG. 13 shows sensor probe 60 and contacts plate 61, according to some embodiments. Sensor probe 60 and contacts plate 61 can be made from a single, flat thin base sheet (matrix sheet, e.g., polyimide). Accordingly, electrodes, conductors, insulators, contact pads, enzyme layer, and other protective layers can be deposited on one side or both sides thereof. Following deposition of materials on the matrix sheet, it is cut (e.g., dye cut or laser cut) to a desired shape and can be folded to receive a final spatial configuration such that the contacts plate 61 is adopted to be received in the sensor housing and the sensor probe (perpendicular to contacts plate) is adopted to be inserted in the subcutaneous tissue. In some embodiments, the contacts plate is configured to provide electrical contact between sensor probe electrodes and the sensor PCBA. The electrodes (at least one) can be deposited on one or both sides of the matrix sheet such that, for example, the working electrode (i.e., glucose sensitive) is deposited on one side and the counter electrode is deposited on the other/second side.

In some embodiments, the working electrode and the counter electrode (and, if required, a reference electrode) are deposited on one side of the matrix sheet. FIG. 13A shows a side view of sensor probe 60 and contacts plate 61 after folding of the matrix sheet, according to some embodiments. Here, contacts plate 61 has 2 sides—contacts plate first side 611 and contacts plate second side 612. In some embodiments, the contacts plate 61 is folded such that contacts plate first side 611 and contacts plate second side 612 are facing the same direction (e.g., upside) (magnified view). FIG. 13A1 shows a top view of the matrix sheet 5 after cutting and before folding, according to some embodiments. Here, matrix sheet 5 includes the sensor probe 60 and contacts palate 61 that is comprised of the contacts plate first side 611, the contacts plate second side 612 (not seen in a top view), and a contacts plate fold 613. FIGS. 13B and 13B1 show two spatial views (mirror images) of the folded matrix sheet that forms the sensor probe 60 and the contacts plate 61, according to some embodiments, and includes electrodes 7 and 8, conductors 71 and 81, and contact pads 72 and 82. Electrode 7, conductor 71, and contact pad 72 can be deposited on one side of the matrix sheet 5; the contact pad 72 can be deposited on the contacts plate second side 612. Electrode 8, conductor 8, and contact pad 82 can be deposited on the second side of the matrix sheet 5; the contact pad 82 can be deposited on contact plate first side 611. In some embodiments, after folding of matrix sheet, contact pad 72 and contact pad 82 face the same direction.

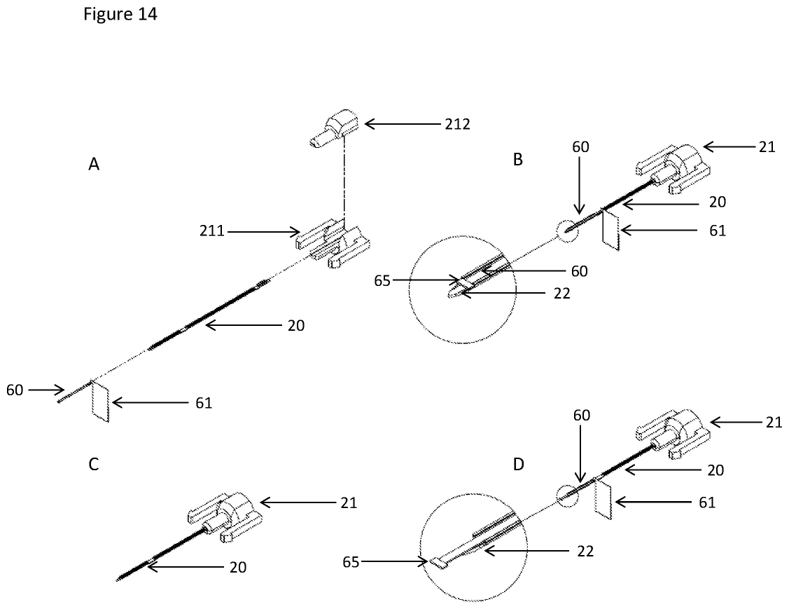

FIG. 14 show spatial views of introducer 20, probe 60, and contacts plate 61, according to some embodiments. FIG. 14A shows an exploded view of introducer 20 and sensor probe 60 before assembly. Accordingly, introducer cap 21 is connected to the introducer 20 at one end (e.g., proximal end) and is comprised of introducer cap snap 211 and introducer cap snap holder 212. The introducer cap snap 211 can be configured to couple with the hammer (e.g., FIGS. 2-6) and drives introducer 20 during insertion. FIG. 14B shows a spatial view of introducer 20, introducer cap 21, sensor probe 60, and contacts plate 61, according to some embodiments, and a magnified view of the introducer tip 22 and sensor probe tip 65 before sensor probe 60 insertion.

FIG. 14C shows a spatial view of introducer 20 and introducer cap 21 before sensor probe insertion, according to some embodiments. FIG. 14D shows a spatial view of introducer 20, introducer cap 21, sensor probe 60, and contacts plate 61, and a magnified view of the introducer tip 22 and sensor probe tip 65 after sensor probe 60 insertion within the subcutaneous tissue and before retraction of introducer 20 (end of the first mounting phase), according to some embodiments.

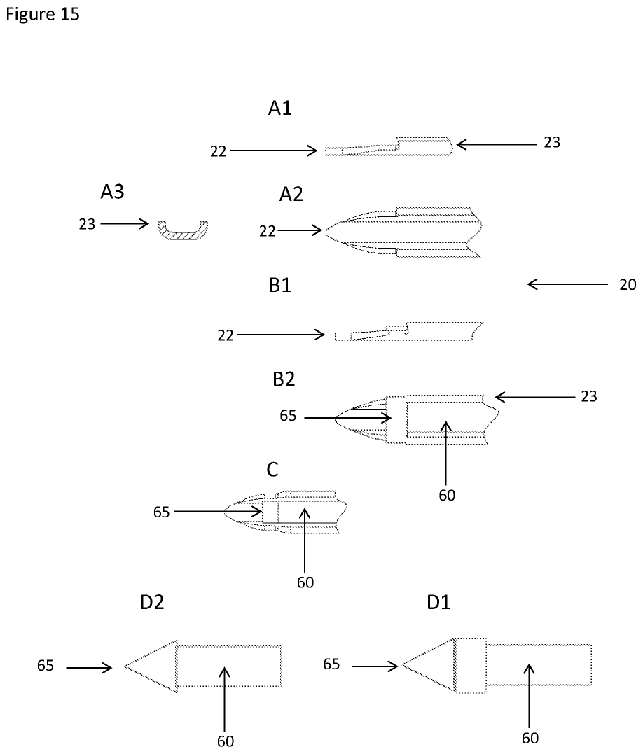

FIG. 15 shows the introducer tip 22 and sensor tip 65, according to some embodiments. FIGS. 15A1-3 show side view (15A1), top view (15A2), and cross-sectional view (15A3) of introducer tip 22, according to some embodiments. Introducer 20, in some embodiments, includes a U-shape cross section having at least one and preferably two longitudinal protrusions: sensor probe holders 23, configured to support sensor probe 60 when it is coupled with introducer 20. FIGS. 15B1-2 show a side view (15B1) and a top view (15B2) of introducer tip 22 and sensor probe tip 60. In some embodiments, sensor tip 65 has a rectangular shape. FIG. 15C shows a top view of a sharp tip 22 of the introducer, and sensor tip 65 of probe 60 (here, sensor tip 65 has a square shape). FIGS. 15D1 and 15D2 show schemes, according to some embodiments, of the senor probe tip having a sharp end.

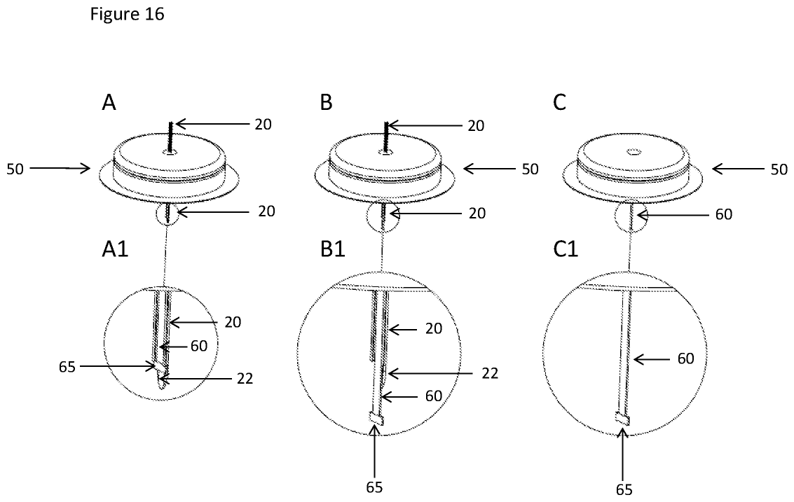

FIGS. 16A-C and 16A1-C1 show spatial (16A-C) and magnified (16A1-C1) views, of the sensor 50, sensor probe 60, introducer tip 22, and sensor tip 65, according to some embodiments, during a first mounting phase and a second mounting phase. Since it is desired to minimize traumatic injury at the tissue surrounding the sensor probe tip and minimize the consequent inflammatory reaction because inflammation reduces the electrodes sensitivity to glucose, in the first mounting phase, the coupled introducer 20 and sensor probe 60 are concomitantly inserted into the subcutaneous tissue. In some embodiments, at the end of forward/first direction displacement of introducer 20 and sensor probe 60, sensor probe 60 is further displaced (e.g., 1-3 mm) in the same (first/forward) direction (i.e., there is a relative movement between introducer 20 and sensor probe 60). Thus, at the end of the first mounting phase, in some embodiments, sensor probe tip 65 is located distally to the introducer tip 22.

The sensor probe 60 is relatively thin 50-100 microns such that and movement within the subcutaneous tissue causes minimal trauma. FIGS. 16A-A1 show sensor probe 60 coupled with introducer 20 before insertion—sensor probe tip 65 resides within the introducer 20. FIGS. 16B-B1 show the introducer tip 22 and the sensor probe tip 65 at the end of the first mounting phase, with the sensor probe tip 65 protruding from the introducer tip 22 (e.g., positioned 1-3 mm apart). FIGS. 14C-C1 show sensor 50 and probe 60 after removal of introducer 20 (not shown) (end of second mounting phase).

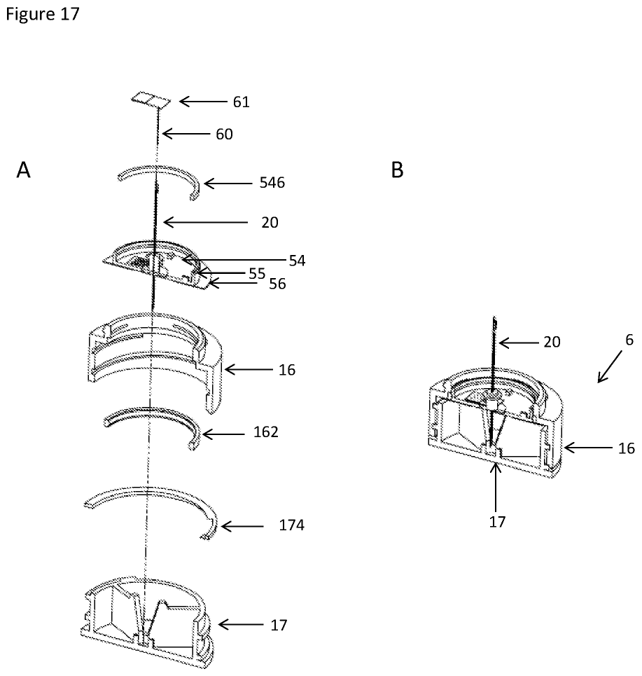

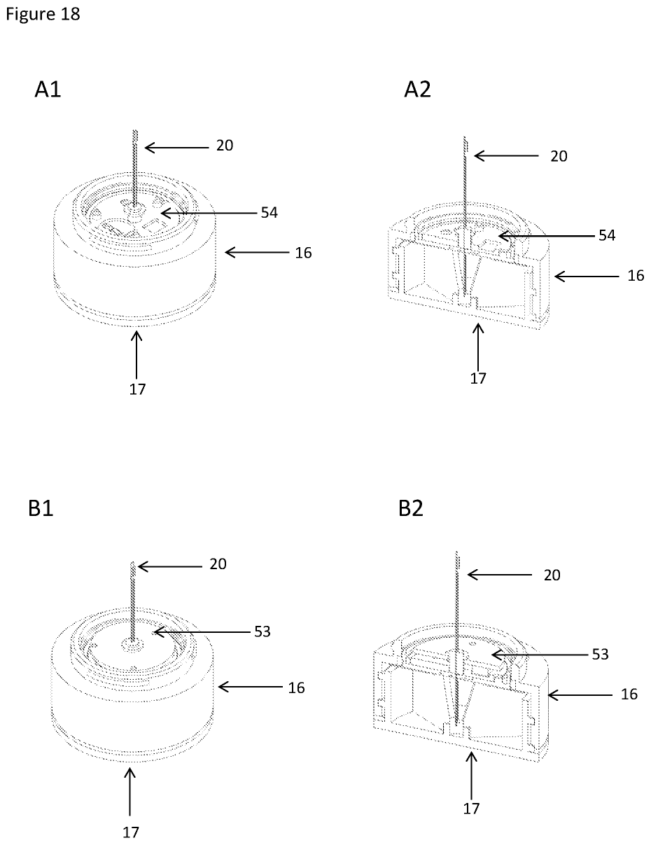

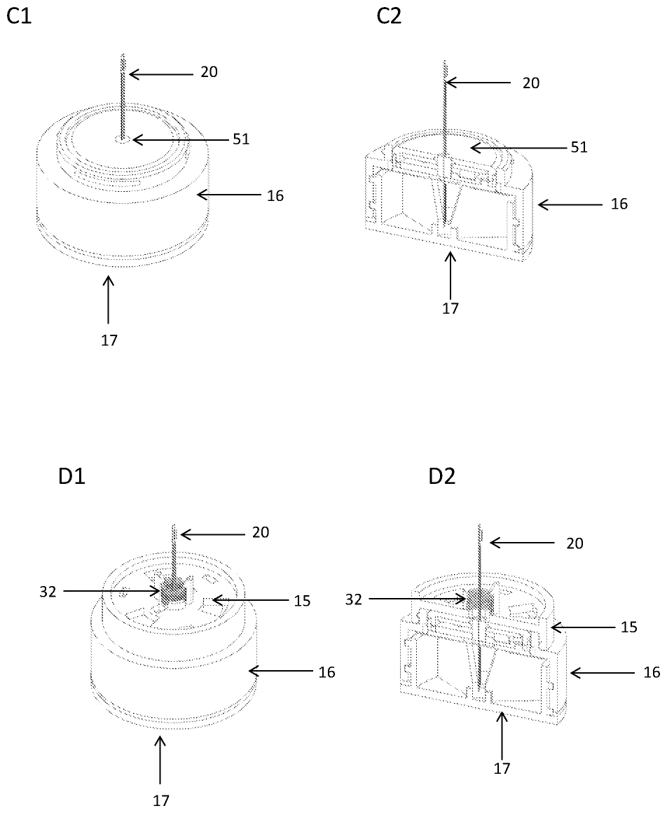

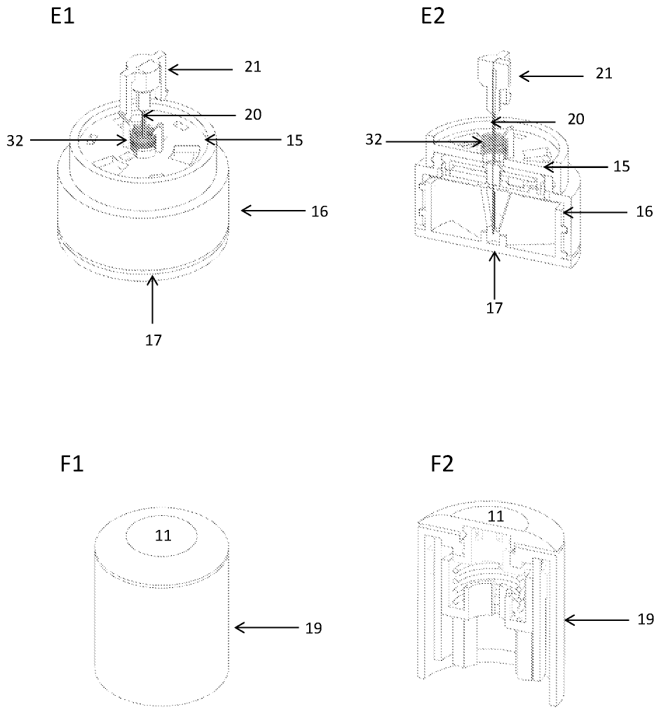

FIGS. 17 and 18 show an assembly process of mounting assembly 1, according to some embodiments. In a first assembly stage, probe compartment 6 is assembled and then sterilized (e.g., gamma, e-beam radiation). The probe compartment 6 is then sealed and provides protection to sensor probe 60 against biologic and chemical contamination. In some embodiments, probe compartment 6 is comprised of at least a portion of mounting assembly 1 and at least a portion of sensor 50 (e.g., FIGS. 1-3). Following sterilization of the probe compartment 6, the non-sterilized spring compartment 9 can be assembled adjacent (e.g., on-top) of sterilized probe compartment 6 (e.g., in a clean room). The spring compartment 9, in some embodiments, comprises at least a portion of mounting assembly 1 and at least a portion of sensor 50 (see e.g., FIGS. 1-3). The introducer 20, according to some embodiments, spans mounting assembly 1 and is configured to be received in both probe compartment 6 and spring compartment 9.

FIGS. 17A-B show exploded view (17A) and oblique cross section view (17B) of the sterilized, sealed compartment (probe compartment 6), according to some embodiments. The probe compartment 6 can include a plurality of, and in some embodiments, all of (parts stacked, e.g., from the bottom-up) protecting lid 17, protecting lid seal 174, probe compartment seal 162, probe compartment housing 16, adhesive 56, sensor housing lower cover 55, sensor base plate, 54, introducer 20, base plate seal 546, sensor probe 60, and contacts plate 61.

FIGS. 18A1-F1 and 18A2-F2 show spatial views (18A1-F1) and oblique views (18A2-F2), respectively, of the various assembly stages of the mounting assembly 1, according to some embodiments. FIGS. 18A1-2 show sterile probe compartment 6 after assembly (e.g., assembly process shown in FIG. 17) and sterilization. The sterile probe/compartment (probe compartment 6) can include a plurality of, and in some embodiments, all of protecting lid 17, probe compartment housing 16, sensor base plate 54, and introducer 20. FIGS. 18B1-2 show the assembled PCBA 53, according to some embodiments, FIGS. 18C1-2 show the assembled sensor housing upper cover 51, according to some embodiments, and FIGS. 18D1-2 show the assembled spring compartment base 15 and the retraction spring 32, according to some embodiments. FIGS. 18E1-2 show the assembled introducer cap 21, according to some embodiments, and FIGS. 18F1-2 show spring compartment housing 19 and insertion spring loaded mechanism (operating button 11, other component are shown in FIGS. 2-6), according to some embodiments.

At a later stage of assembly, according to some embodiments, spring compartment housing 19 can be rigidly connected to probe compartment housing 16 forming mounting assembly 1 (as shown in FIGS. 1-6). In some embodiments, assembled mounting assembly 1 can be sterilized with a gas (e.g., ethylene oxide), and sealed probe compartment 6, which can be pre-sterilized with radiation (e.g., gamma or e-beam), protects the gas sensitive enzyme (deposits on the probe 60) from potential damage of the gas. In such a configuration, both compartments (probe compartment 6 and spring compartment 9) can be sterilized as well as all components of the sensor 50 (including PCBA).

While various inventive embodiments have been described and illustrated herein, those of ordinary skill in the art will readily envision a variety of other means and/or structures for performing the function, and/or obtaining the results and/or one or more of the objects/advantages described herein, and each of such variations and/or modifications is deemed to be within the scope of the inventive embodiments described herein. More generally, those skilled in the art will readily appreciate that all parameters, dimensions, materials, steps, and configurations described herein are meant to be merely an example and that the actual parameters, dimensions, materials, steps, and configurations will depend upon the specific application or applications for which the inventive teachings is/are used. Those skilled in the art will recognize, or be able to ascertain using no more than routine experimentation, many equivalents to the specific inventive embodiments described herein. It is therefore to be understood that the foregoing embodiments are presented by way of example only and that, within the scope of claims supported by the subject disclosure and equivalents thereto, and inventive embodiments may be practiced otherwise than as specifically described and claimed. Inventive embodiments of the present disclosure are directed to each individual feature, device, system, article, material, kit, step, function/functionality, and method described herein. In addition, any combination of two or more such features, devices, systems, articles, materials, kits, steps, functions/functionality, and methods, if such features, systems, articles, materials, kits, steps, functions/functionality, and methods are not mutually inconsistent, is included within the inventive scope of the present disclosure, and considered embodiments.

Embodiments disclosed herein may also be combined with one or more features, as well as complete systems, devices, and/or methods, to yield yet other embodiments and inventions. Moreover, some embodiments, may be distinguishable from the prior art by specifically lacking one and/or another feature disclosed in the particular prior art reference(s); i.e., claims to some embodiments may be distinguishable from the prior art by including one or more negative limitations.

Also, as noted, various inventive concepts may be embodied as one or more methods. The acts performed as part of the method(s) may be ordered in any suitable way. Accordingly, embodiments may be constructed in which acts are performed in an order different than illustrated, which may include performing some acts simultaneously, even though shown as sequential acts in illustrative embodiments.

Any and all references to publications or other documents, including but not limited to, patents, patent applications, articles, webpages, books, etc., presented anywhere in the present application, are herein incorporated by reference in their entirety. Moreover, all definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The terms “can” and “may” are used interchangeably in the present disclosure, and indicate that the referred to element, component, structure, function, functionality, objective, advantage, operation, step, process, apparatus, system, device, result, or clarification, has the ability to be used, included, or produced, or otherwise stand for the proposition indicated in the statement for which the term is used (or referred to).

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is currently claimed:

1. A continuous glucose monitoring system for continuously monitoring glucose levels of a user, the system comprising:
a mounting assembly; and
a sensing device, wherein at least a portion of the sensing device is housed within the mounting assembly prior to mounting the sensing device on the user.

2. The system of claim 1, wherein the sensing device includes at least a transmitter configured to transmit signals corresponding to sensed glucose levels of the user.

3. The system of claim 2, wherein the system further comprising a remote display unit configured with at least with a receiver for receiving the sensed glucose readings from the sensing device via the transmitter.

4. The system of any of claims 1-3, wherein the sensing device comprises a sensor.

5. The system of any of claims 1-3, wherein the sensing device comprises a sensor, and a sensor probe configured for insertion into the subcutaneous tissue of the user.

6. The system of claim 2 or 3, wherein the sensing device comprises a sensor configured for adhering to the skin of the user, and a sensor probe configured for insertion into the subcutaneous tissue of the user.

7. The system of claim 5 or 6, wherein the sensor optionally includes a lower portion having an adhesive for removable affixation of the sensor to the skin of the user.

8. The system of any of claims 5-7, wherein the sensor includes a sensor housing.

9. The system of any of claims 5-8, wherein the sensor includes a sensor control unit.

10. The system of claim 8, wherein the sensor probe includes a first portion configured for insertion into subcutaneous tissue, and a second portion configured for receiving by the sensor housing.

11. The system of claim 9, wherein the sensor probe includes a first portion configured for insertion into subcutaneous tissue, and a second portion configured for receiving by the sensor housing.

12. The system of any of claims 8-11, wherein the sensor housing comprises an upper cover and a lower cover.

13. The system of claim 12, wherein the lower cover includes an adhesive for adhering the sensor to the skin of the patient.

14. The system of any of claims 5-13, wherein the sensor includes a printed circuit board assembly (PCBA).

15. The system of any of claims 5-14, wherein at least one of the sensor housing and the PCBA also includes at least one of a receiver, a transmitter, a processing circuit, a battery, an alarm system, and data storage means.

16. The system of any of claims 5-14, wherein at least one of the sensor housing and the PCBA also includes at least two of a receiver, a transmitter, a processing circuit, a battery, an alarm system, and data storage means.

17. The system of any of claims 5-14, wherein at least one of the sensor housing and the PCBA also includes a receiver, a transmitter, a processing circuit, a battery, an alarm system, and data storage means.

18. The system of any of claims 15-17, wherein the receiver and the transmitter comprise a transceiver.

19. The system of any of claims 5-18, wherein the sensor includes at least one, and preferably, a plurality of conductive contacts configured for coupling to one or more respective contact pads of the sensor probe.

20. The system of any of claims 5-19, wherein the sensor probe includes at least of: at least one working electrode, a counter electrode, at least two (2) electrical conductors, and at least two (2) contact pads.

21. The system of any of claims 5-19, wherein the sensor probe includes at least one working electrode, a counter electrode, at least two (2) electrical conductors, and at least two (2) contact pads.

22. The system of any of claims 5-21, wherein the sensor probe is configured with a planar shape, and includes at least two (2) electrodes.

23. The system of claim 22, wherein the at least two (2) electrodes comprises at least a working electrode.

24. The system of claim 23, wherein the working electrode is configured for positioning on at least one of a first side of the sensor probe and at a distal end thereof.

25. The system of any of claims 22-24, wherein the at least two (2) electrodes comprise a counter electrode.

26. The system of claim 22, wherein the at least two (2) electrodes comprise at least a working electrode and a counter electrode, wherein the working electrode is configured for positioning on at least one of a first side of the sensor probe and at a distal end thereof, and the counter electrode is configured for arrangement on a side of the sensor probe opposite to the side where the working electrode is positioned on the sensor probe.

27. The system of any of claims 22-26, wherein each electrode is connected via electrical conductors to a respective contact pad.

28. The system of claim 27, wherein one or more contact pads are configured for positioning on at least one side of a contacts plate, the contacts plate optionally included with the sensor probe.

29. The system of claim 27, wherein one or more contact pads are configured for positioning on each side of a contacts plate, the contacts plate optionally included with the sensor probe.

30. The system of claim 28 or 29, wherein the contacts plate is positioned approximately perpendicular to the sensor probe.

31. The system of claim 30, wherein the contacts plate is positioned within the sensor housing.

32. The system of claim 31, wherein upon the contacts plate being positioned within the sensor housing, at least one (1) contact pad is facing the PCBA, and optionally, another (e.g., the other) contact pad is facing an opposite direction.

33. The system of claim 32, wherein the contacts plate is folded on a first side at 180 degrees, such that each contact pad is facing the PCBA.

34. The system of any of claims 29-33, wherein the sensor probe and the contacts plates are constructed from a sheet configured such that the sensor probe is perpendicular to the skin, the contacts plate is perpendicular to the sensor probe (parallel to skin), and both contact pads are facing the PCBA.

35. The system of claim 34, the sheet comprises a single matrix sheet.

36. The system of any of claims 2-35, wherein the mounting assembly is configured for mounting the sensing device onto a patient.

37. The system of any of claims 5-36, wherein the mounting assembly is assembled with the sensing device during manufacture, such that it is ready to mount the sensing device onto a patient.

38. The system of any of claims 1-37, wherein the mounting assembly comprises a first, non-sealed, non-sterilized compartment, and a second sealed, sterilized compartment.

39. The system of claim 38, wherein at least one of the first and second compartments each include a housing.

40. The system of claim 38 or 39, wherein the first compartment is arranged immediately adjacent the second compartment.

41. The system of any of claims 38-40, wherein the first compartment includes at least one of an insertion means and a retraction means.

42. The system of any of claims 38-41, wherein the first compartment includes an insertion means and a retraction means.

43. The system of any of claims 38-42, wherein the second compartment includes at least one of the sensor probe and a portion of the sensor.

44. The system of any of claims 38-43, wherein the second compartment includes the sensor probe and a portion of the sensor, and optionally an elastomeric septum configured to sealing the second compartment.

45. The system of any of claims 38-44, wherein the second compartment includes a protection lid.

46. The system of claim 45, wherein a side of the second compartment from which the protection lid is removed in configured for placement adjacent to the skin of the patient.

47. The system of any of claims 38-46, wherein upon mounting the sensing device, at least one of the sensor and sensor probe are displaced within the second compartment and mounted onto the patient.

48. The system of any of claims 38-46, wherein upon mounting the sensing device, the sensor and sensor probe are displaced within the second compartment and mounted onto the patient.

49. The system of any of claims 5-52, further comprising a sensor-probe introducer (“introducer”) configured to insert the sensor probe into the subcutaneous tissue of the patient.

50. The system of claim 49, wherein the introducer comprises:
a sharp tip at a first end configured to penetrate the skin of the patient, and
an introducer cap, at a second end configured to drive the introducer in a first direction during insertion and a second direction opposite the first direction during retraction.

51. The system of claim 49 or 50, wherein the introducer is positioned within the mounting assembly.

52. The system of claim 49 or 50, wherein the introducer is positioned within the mounting assembly and spans the first compartment, the second compartment, and the elastomeric septum.

53. The system of claim 52, wherein the introducer is configured for displacement through the septum in a first direction during sensor probe insertion, and in a second direction opposite to the first direction during introducer retraction.

54. The system of any of claims 5-53, wherein the mounting assembly is configured to automatically operate, including automatically adhering the sensor to the patient's skin, inserting the sensor probe into the subcutaneous tissue, and retracting the sensor probe introducer.

55. The system of claim 54, further comprising a trigger configured to initiate the automatic operation of the mounting assembly to mount the sensing device on the user.

56. The system of claim 55, wherein the trigger includes an operating button or an operating switch configured to receive user or operator input.

57. The system of any of claim 41-56, wherein the insertion means comprises a spring loaded driving mechanism configured to mount the sensing device onto the patient.

58. The system of any of claims 41-57, wherein the retraction means comprises a spring loaded retraction mechanism for removing the introducer while leaving the sensing device on the patient.

59. The system of any of claims 57-58, wherein upon pressing a/the operating button, the trigger releases the insertion spring of the insertion means that drives the sensing device, including the sensor, the sensor probe, and optionally the introducer, in a first direction, thereafter, the retraction spring is released and the introducer is retracted in a second opposite direction such that the sharp end of the introducer is at least substantially concealed within the mounting unit.

60. The system of any of claims 49-59, wherein the introducer comprises a rigid planar structure including a/the sharp tip, and is adapted to support the sensor probe during insertion.

61. The system of any of claims 49-59, wherein the introducer and sensor probe are concomitantly inserted into the subcutaneous tissue.

62. The system of claim 61, wherein at the end of insertion, the sensor probe is further advanced in the first direction (e.g., 1-3 mm).

63. The system of any of claims 38-62, wherein both the first and the compartments are sterilized compartments.

64. The system of claim 63, wherein the first compartment is sterilized first with an e-beam and/or gamma radiation, followed by the second compartment which is sterilized with a gas (e.g., ethylene oxide).

65. The system of any of claims 49-64, wherein the introducer is planar in cross-section.

66. The system of any of claims 49-65, wherein the introducer is not oval or round in cross-section.

67. The system of any of claims 49-66, wherein the introducer includes a u-shaped cross-section.

68. The system of any of claims 49-68, wherein the introducer includes at least one and preferably two shoulders configured to at least one of secure the sensor probe in insertion and allow introducer retraction, while leaving the sensor probe in the body.

69. A glucose monitoring sensing device mounting assembly according to any of claims 1-68.

70. A glucose monitoring sensing device according to any of claims 1-68.

71. A glucose sensor according to any of claims 1-68.

72. A glucose sensor probe according to any of claims 1-68.

73. An assembly method for assembling a mounting assembly and a sensing device for a continuous glucose monitoring system, comprising assembling the sensing device, comprising:
depositing glucose sensitive layers, electrical conductors, insulators, and contact pads on one or both sides of a matrix sheet, such depositing includes forming one or more electrodes on the matrix sheet;
cutting and/or folding the matrix sheet such that it is configured to include the sensor probe and the contacts plate;
assembling of the sensor contacts plate, sensor printed-circuit-board-assembly (PCBA), and sensor housing as to form a sensor, assembling the mounting assembly, comprising:
assembling at least one of an insertion spring and a retraction spring into a first compartment of the mounting assembly;
assembling and sterilizing a second compartment of the mounting assembly; and
placing at least a portion of the sensing device in the second compartment.

74. The method of claim 73, wherein both the first and the second compartments are sterilized.

75. The method of claim 74, wherein the second compartment is sterilized first with an e-beam and/or gamma radiation, followed by the first compartment which is sterilized with a gas (e.g., ethylene oxide).

76. The method of any of claims 73-75, wherein at least one of the probe and contacts plate are formed on/from the matrix sheet.

77. The method of any of claims 73-76, wherein the sensor probe and the contacts plate are formed on/from the matrix sheet.

78. The method of claim 76 or 77, wherein at least one of the sensor probe and the contacts plate is formed on/from the matrix sheet via cutting and/or folding so as to form an/the appropriate spatial configuration.

79. The method of any of claims 73-78, wherein at least one of the one or more electrodes, one or more conductors, and one or more contact pads, are positioned on at least one side of the matrix sheet.

80. The method of any of claims 73-78, wherein at least one of the one or more electrodes, one or more conductors, and one or more contact pads, are positioned on both sides of one matrix sheet.

81. The method of claim 80, wherein the folding of contacts plate is configured to arrange the contact pads to face the same direction.

82. The method of claim 81, wherein the same direction comprises towards the PCBA.

83. A device, system, and/or method according to any of the embodiments disclosed herein.
